(19) United States
(12) Patent Application Publication
(10) Pub. No.: US 2026/0306137 A1
(43) Pub. Date: Oct. 8, 2026

(54) BALLOON CERVICAL DILATION DEVICES WITH TELESCOPING INNER SHAFT, METHODS OF USE AND MANUFACTURE AND KITS THEREFOR

(71) Applicant: MEDICINES360, San Francisco, CA (US)

(72) Inventors: Rob DECKMAN, San Bruno, CA (US); Mitchell David CREININ, Davis, CA (US); Bryce IGO, Flagstaff, AZ (US); Devon MARTINDALE, Flagstaff, AZ (US); Alberto QUINTERO RETIS, Flagstaff, AZ (US); Benjamin Michael TRAPP, Scottsdale, AZ (US)

(73) Assignee: MEDICINES360, San Francisco, CA (US)

(21) Appl. No.: 19/629,472

(22) Filed: Mar. 26, 2026

Related U.S. Application Data
(60) Provisional application No. 63/878,282, filed on Sep. 9, 2025.
(60) Provisional application No. 63/783,418, filed on Apr. 4, 2025.

(51) Int. Cl. A61B 17/42; A61M 29/02; A61B 17/00; A61B 17/02; A61B 17/12; A61M 25/00; A61M 25/10

(52) U.S. Cl. CPC A61B 17/4241; A61M 29/02; A61B 2017/00991; A61B 2017/0225; A61B 2017/12004; A61B 2017/4225; A61M 2025/0004; A61M 25/0041; A61M 25/1011; A61M 25/10182; A61M 25/10186; A61M 25/1034; A61M 2025/105; A61M 2025/1061

(57) ABSTRACT

Balloon cervical dilation devices are provided that include an elongated flexible tubular member carrying one or more inflatable balloons and, in certain embodiments, a telescoping inner shaft configured to resist kinking under tension. The devices may incorporate a flow restrictor between a dilation balloon and a proximal fluid reservoir to enable controlled, gradual inflation for cervical ripening or uterine compression while reducing patient pain. In some implementations, a single device can be used sequentially for cervical dilation and postpartum hemorrhage management. The balloons can be expanded, for example, up to about 30 mm to dilate the cervical canal or compress the uterus. Also disclosed are methods of using the devices and kits including the devices and associated components.

### CROSS-REFERENCE

This application claims the benefit of U.S. Provisional Application No. 63/783,418, filed Apr. 4, 2025, and Provisional Application No. 63/878,282, filed Sep. 9, 2025, which applications are incorporated herein in their entirety by reference.

### BACKGROUND

### Field

The disclosure relates to the field of obstetrics and gynecology. More specifically the disclosure relates to cervical dilation devices for dilation or compression and methods.

### Background

Cervical dilation tools are used by healthcare professionals to access the cervix 32. For context, FIG. 1 illustrates the female uterine anatomy. The uterus 30 is accessed via the cervical canal 33 of the cervix 32 which is located partially within the vagina 34. Devices are inserted into the uterus 30 by advancing the cervical dilation device in a distal direction for insert 20, and removed by withdrawing the cervical dilation device in a proximal direction to remove 10.

The use of cervical dilation tools is, for example, indicated when there is a need to insert an instrument through the cervical canal 33 and the cervical canal 33 is not dilated adequately to allow free passage of an instrument. In one process, a vaginal speculum is used to hold the vagina 34 apart followed by inserting a series of sticks or rods called osmotic dilators to open the cervix slowly. The osmotic dilator can be formed from, for example, an open-porosity polyvinyl alcohol sponge loaded with magnesium sulfate and compressed into a rod. The osmotic dilator is used to slowly dilate and soften the cervix and operates by increasing in size as the dilator absorbs fluid from the surrounding patient tissue. Inserting dilators can be very uncomfortable and often causes cramping.

Another condition that can arise for women is postpartum hemorrhage (PPH). PPH is a serious condition that occurs when there is excessive blood loss after giving birth. PPH is a leading cause of maternal mortality worldwide. PPH can be treated, for example, with medicine, a balloon (e.g., uterine balloon tamponade) or other device to compress the uterus.

What is needed is an easy-to-use and easy-to-control cervical dilation device that is operable to dilate the cervical canal over time that reduces pain for the patient and/or compress the uterus in the event of excessing blood loss.

### SUMMARY

Disclosed are easy-to-use and easy-to-control cervical dilation devices operable to dilate the cervical canal that reduces pain for the patient and/or compress the uterus in the event of excessing blood loss. The cervical dilation devices have an elongated flexible member and one or more balloons. A flow restrictor can be provided between a dilation balloon and a proximal reserve. In some configurations, the same device can be used to dilate the cervix and then again for compression. Methods of using the disclosed devices and kits are also disclosed.

Also disclosed are cervical dilation devices that include a telescoping inner shaft which is operable to prevent kinking when the balloon is subjected to tension. The disclosed cervical dilation devices may include an inflatable balloon with a proximal balloon end and a distal balloon end, the inflatable balloon may be operable to expand to an expanded diameter up to 30mm.

Both the foregoing general description and the following detailed description are exemplary and explanatory only and are not restrictive of the disclosed examples.

### INCORPORATION BY REFERENCE

All publications, patents, and patent applications mentioned in this specification incorporated by reference herein to the same extent as if each individual publication, patent, or patent application was specifically and individually indicated to be incorporated by reference to the maximum extent allowable by law, except for any definitions, disclaimers, disavowals, and inconsistencies.

CN 108742800 B issued 11/6/2018 by Luo et al. for Cervical dilation apparatus;

CN 108744237 A published 11/6/2018 by Zhang et al. for Cervical dilation equipment;

CN 109847174 A published 6/7/2019 by Zhao et al. for A kind of genital tract extension fixture for the stream of people and induced labor;

CN 201353186 Y published 12/2/2009 by Zheng for Cervical dilation device;

CN 204932561 U published 1/6/2026 by Chen for Cervical dilator;

CN 204971508 U published 1/20/2016 by Ruiming for Two sacculus cervix dilators;

CN 211273075 U published 8/18/2020 by Zhaolin for Cervical dilation device;

EP 0193406 A2 published 9/3/1986 by Brennan et al. for Device for Controlling Incontinence;

EP 2742966 A1 published 6/18/2014 by Wu et al. for Application Of Water-Absorption Material In Medical Cavity Channel Expander;

US 1,735,519 A issued 11/12/1929 by Vance for Physician’s Dilator;

US 3,848,602 A issued 11/14/1974 by Gutnick for Abortion Facilitating Device and Process;

US 3,900,033 A issued 8/19/1975 by Leininger et al. for Dilator for Cervical Canal;

US 4,137,922 A issued 2/6/1979 by Leininger et al. for Dilator for Cervical Canal;

US 4,228,801 A issued 10/21/1980 by Magnasco et al. for Dilator for Application in Medical Field;

US 4,348,893 A issued 12/9/1980 by Michaels for Cervical Dilator;

US 4,480,642 A issued 11/6/1984 by Stoy et al. for Dilation Device for the Cervix;

US 5,065,772 A issued 11/19/1991 by Cox Jr. for Inflatable Cervical Pessary;

US 5,947,991 A issued 9/7/1999 by Cowan for Single Balloon Device for Cervix;

US 5,947,992 A issued 9/7/1999 by Zadini et al. for Inflatable Menstrual Cup for Blood Leakage Prevention;

US 8,097,014 B2 issued 1/17/2012 by Borkon for Variable rigidity vaginal dilator and use thereof;

US 9,629,644 B2 issued 4/25/2017 by Schreck et al. for Devices and methods for dilating a paranasal sinus opening and for treating sinusitis;

US 2006/0200187 A1 published 9/7/2006 by Guide for Childbirth inflatable water/air vaginal obturator;

US 2006/178692 A1 published 8/10/2006 by Condrea et al. for Dilator For Body Passageway;

US 2020/048640 A1 published 2/6/2020 by Stoy et al. for Combined Osmotic And Hydrogel Cervical Dilators And Method Of Making Same;

US 2023/0293204 A1 published 9/21/2023 by Reeves for Cervical Dilator;

WO 81/01098 A1 published 4/30/1981 by Zon for An Armoured Balloon For Cervical Dilatation And Cervical Resistance Measurement;

WO 2013/020435 A1 published 2/14/2013 by Wu et al. for Application Of Water-Absorption Material In Medical Cavity Channel Expander;

WO 2023/023582 A1 published 2/23/2023 by Reeves for Cervical Dilator; and

Cook® Cervical Ripening Balloon with Style, available from https://www.cookmedical.com/products/wh_crbs_webds/.

### BRIEF DESCRIPTION OF THE DRAWINGS

The novel features of the teachings are set forth with particularity in the present disclosure. As will be appreciated by those skilled in the art, the various features illustrated in the drawings may not be drawn to scale. Moreover, the illustrations presented in the present disclosure are not meant to be actual views of any particular apparatus (e.g., device, system, etc.) or method, but are merely representations that are employed to describe various examples of the disclosure. Accordingly, the dimensions of the various features may be arbitrarily expanded or reduced in the figures for clarity. In addition, some of the drawings may be simplified for clarity. Thus, the drawings may not depict all of the components of a given apparatus (e.g., device) or all operations of a particular method. The novel features of the disclosure are set forth with particularity in any claim presented.

A better understanding of the features and advantages of the present disclosure will be obtained by reference to the following detailed description that sets forth illustrative examples, in which the principles of the disclosed teachings are utilized, and the accompanying drawings of which:

FIG. 1 illustrates female uterine anatomy for operational context of the disclosed cervical dilation devices;

FIGS. 2A-D illustrate an exemplar cervical dilation device with a balloon and an elongated flexible tubular member in an unexpanded configuration, an anchor configuration, and dilation configurations;

FIGS. 3A-H illustrate additional details of an exemplar cervical dilation device;

FIGS. 4A-C illustrate a flow restrictor incorporatable in the cervical dilation device;

FIGS. 5A-D illustrate an exemplar cervical dilation device;

FIGS. 6A-G illustrate an exemplar cervical dilation device with a telescoping inner shaft;

FIGS. 7A-G illustrate an exemplar process of operating the cervical dilation device;

FIGS. 7H-M illustrate a process of operating and inflating the cervical dilation device; and

FIG. 8 illustrates a kit for a cervical dilation device.

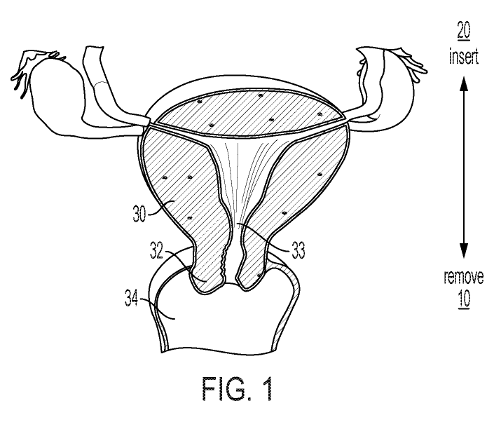

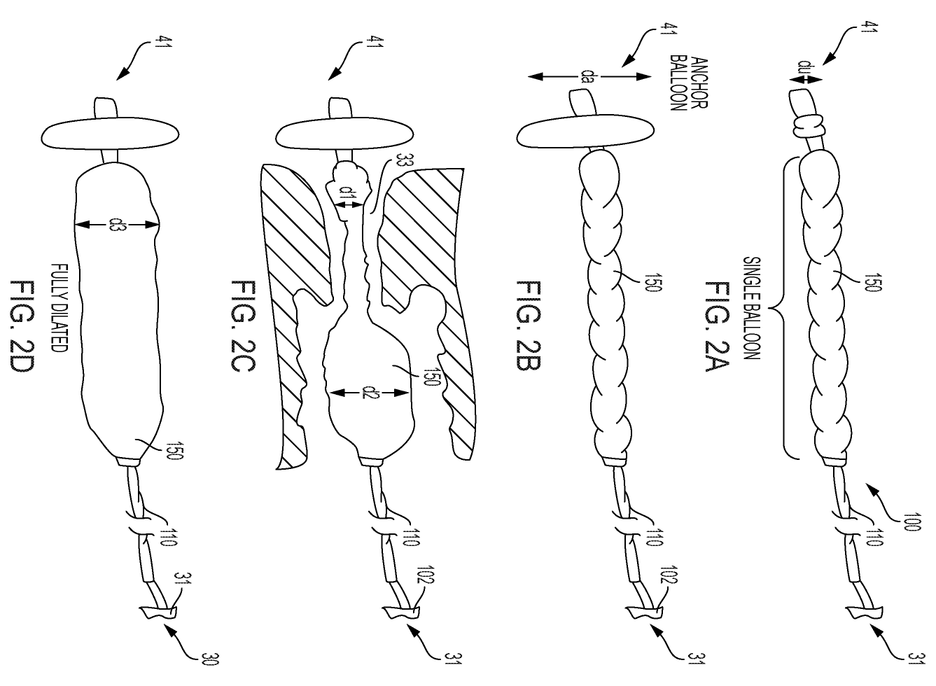

### DETAILED DESCRIPTION

### Devices

FIG. 2A illustrates an exemplar cervical dilation device 100 with a dilation balloon 150 and an elongated flexible tubular member 110, such as a catheter, in an unexpanded configuration. The elongated flexible tubular member 110 can have a gentle curve at the distal end to improve insertion. The cervical dilation device 100 is suitable for cervical dilation or cervical compression The cervical dilation device 100 has a length L and a width W. At least one fluid fitting 102, such as a Luer Lock with a check valve, is provided at a proximal 31 position on the elongated flexible tubular member 110. The dilation balloon 150 can have three operable sections: a distal anchor section (e.g., distal balloon 152), a proximal cervix section, and a cervical canal section between the distal anchor section and the proximal cervix section. The three operable sections of the balloon can be integrally formed as a single element (e.g., a single balloon) or to operate as a single element. As shown in FIG. 2B, as the cervical dilation device 100 begins to dilate (anchor dilation step), a distal balloon 152 located at the distal anchor section, begins to expand in diameter from an unanchored diameter dU to an anchored diameter dA. As shown in FIG. 2C, during additional dilation (target dilation step), a proximal cervix section mostly residing in the vaginal canal of the dilation balloon 150 begins to change from a first diameter d1 to a second diameter d2. With additional dilation, as shown in FIG. 2D, a distal end 41 of the dilation balloon (or intermediate portion of the dilation balloon positioned between the distal anchor section and the proximal cervical canal section) changes from the first diameter d1 to a second diameter d3. The second diameter d3 in the distal portion of the dilation balloon 150 can be the same as the second diameter d2 of the proximal portion of the dilation balloon 150, or a different diameter as needed.

Marker bands, or small thin-walled tubes, can be incorporated at one or more locations along the length of the dilation balloon 150 to enhance visibility and aid with placement of the dilation balloon 150 during use. The marker bands are visible when imaging techniques like fluoroscopy are used. Marker bands can be made from any suitable radiopaque material, such as gold, platinum, tantalum, or their alloys. Polyurethan bands impregnated with a radiopaque material, such as tungsten or platinum, can also be used in any of the disclosed configurations. In some configurations, a first marker band is placed 5 mm from the proximal end 31 of the balloon and a second marker band is placed 15 mm from the proximal end of the balloon (i.e., 10 mm distally from the first marker band). The marker bands facilitate determining where the balloon is positioned. If the distal marker band is visible, then the dilation balloon needs to be advanced further in the distal direction. When the distal marker band is not visible and the proximal marker band remains visible, then the dilation balloon has been advanced distally a correct distance. If the proximal marker band is no longer visible then the dilation balloon has been advanced to far proximally or the dilation balloon is being drawn in a distal direction.

In some examples, the cervical dilation device 100 can be provided with three separate balloons: a distal balloon, a dilation balloon, and a proximal reservoir balloon. In other configurations, only two balloons are provided: a distal balloon and a dilation balloon (which can also function as a reservoir balloon in some examples). In another configuration, only a single dilation balloon is provided with a specifically shaped exterior profile that allows the single balloon to have more than one function during use (e.g., anchoring and dilation). As will be appreciated by those skilled in the art, in some examples, no reservoir is provided and inflation of the cervical dilation device 100 is achieved via an insufflator that is operable to deliver gas or fluid to an interior cavity of the cervical dilation device 100. In some configurations, a catheter only is provided with a pressure reservoir either internal or external to the body.

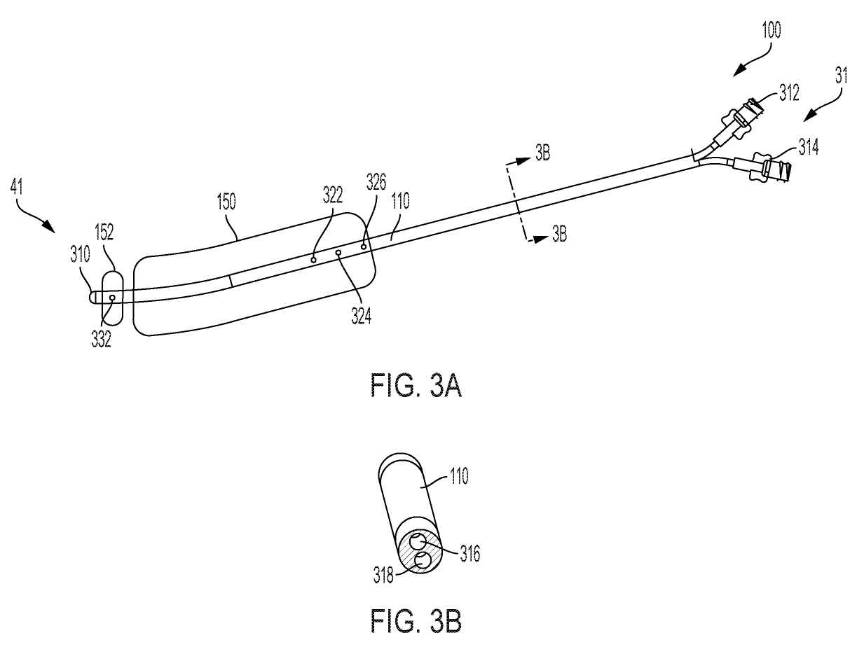

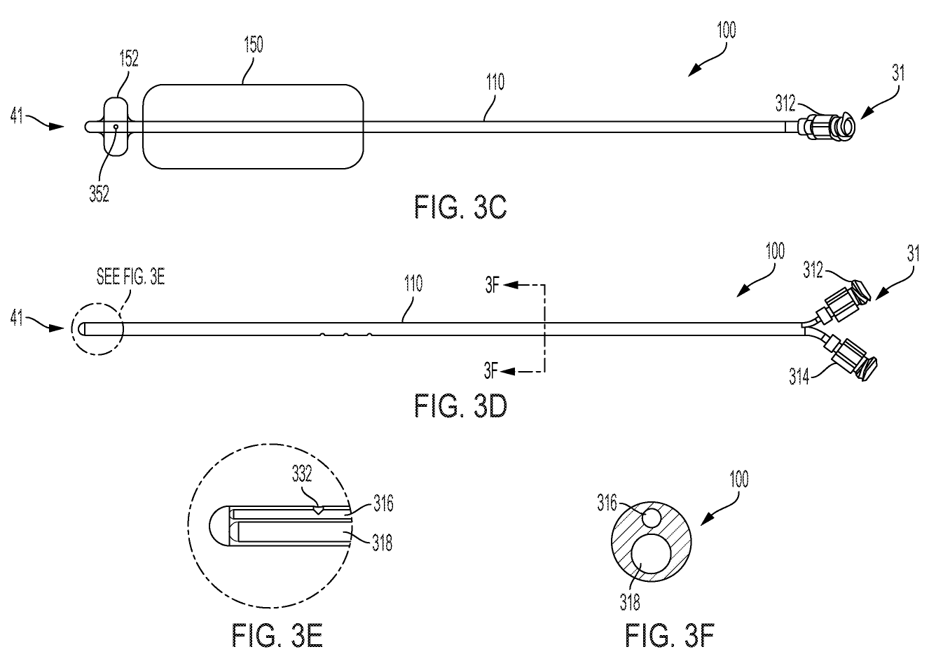

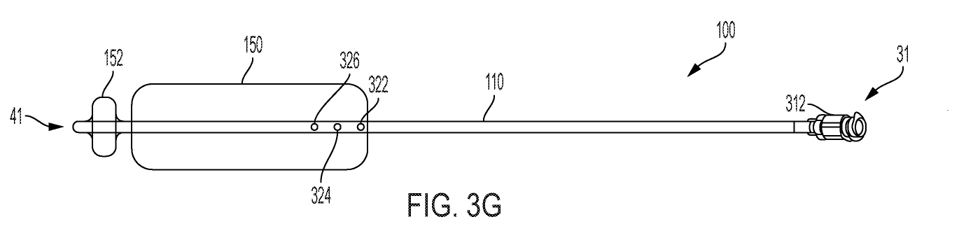

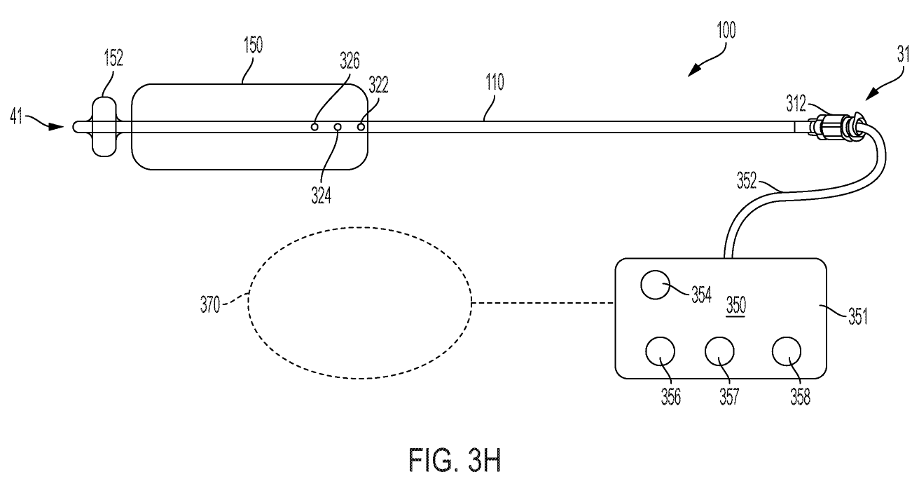

FIGS. 3A-G illustrate additional details of an exemplar cervical dilation device 100. As shown in FIG. 3A, the cervical dilation device 100 has a length of from 200 mm to 300 mm, in a specific example 238 mm, and a flexible distal end. The distal end of the cervical dilation device 100 can also be a rounded distal end 101. The width of the cervical dilation device 100 ranges from 3.0 mm to 7 mm, in a specific example 5.0 mm. The cervical dilation device 100 is illustrated with two fluid fittings 312, 314 at a proximal end 31. The two fluid fittings 312, 314 are in communication with a corresponding channel 316, 318 (shown in FIG. 3B) that traverses substantially the entire length of the elongated flexible tubular member 110. The fluid channels 316, 318 are in fluid communication with either the proximal fill port(s) or the distal fill port(s).

A distal balloon 152 is provided towards or at the distal end 41 of the elongated flexible tubular member 110. The distal balloon 152 can have a first diameter and a second dilated diameter. The second diameter of the distal balloon 152 can be from 15 mm to 25 mm in diameter, in a specific example 25 mm. The distal balloon 152 can be a compliant balloon that continues to expand as pressure within the balloon increases. In an alternative example, the distal balloon 152 can be non-compliant with a fixed final shape.

At least one distal fill port 332 is provided along the length of the elongated flexible tubular member 110 at the distal end 41 in communication with one of the fluid channels. The distal fill port 332 can have a diameter of from 1.0 mm to 2.0 mm, in a specific example 1.5 mm. The distal fill port 332 is operable to fill a distal balloon 152 during use. Suitable balloon materials are biocompatible and able to stretch 100%-800% to achieve a desired inflated diameter for the balloon. For example, suitable material for the distal balloon is silicone or polyurethane material. The distal balloon 152 can be filled with any suitable material including, for example, sterile saline solution.

A dilation balloon 150 is provided proximal to the distal balloon 152. The dilation balloon 150 can have a first diameter and a second diameter. The first diameter can be a starting diameter and the second diameter can be a diameter achievable after inflation. The second diameter can be from 25 mm to 35 mm, in a specific example 30 mm. The dilation balloon 150 can be non-compliant so that it expands only to a target size or to a predetermined size even if pressure continues to rise as a result of inflation efforts. One or more proximal fill ports 322, 324, 326 is operable to fill the dilation balloon 150. The one or more proximal fill ports 322, 324, 326 can have a diameter of from 1.0 mm to 2.0 mm, in a specific example 1.5 mm. Where more than one proximal fill ports are provided, the ports can be spaced from 5 mm to 15 mm apart, in a specific example 10 mm apart. Moreover, the one or more proximal fill ports 322, 324, 326 can be in communication with a second channel 318.

FIG. 3B illustrates a perspective taken at section, from 3B-3B of FIG. 3A, two fluid channels 316, 318 are provided. The fluid channels 316, 318 can have a consistent diameter along the length of the elongated flexible tubular member 110. The diameter of each of the fluid channels 316, 318 can be the same or different and range from .02 mm to 1.0 mm. In one example, the diameter for the first fluid channel 316 is 0.039 mm, and the diameter for the second channel 318 is 0.079 mm. The distal tip 310 of the flexible tubular member can be echogenic to enhance visibility during ultrasound procedures by reflecting sound waves. The use of an echogenic tip can be useful to track the location of the cervical dilation device 100 during use. FIG. 3C illustrates the cervical dilation device 100 without any curvature of the elongated flexible tubular member 110. FIG. 3D illustrates the flexible tubular member 110 without the balloons. FIG. 3E is an enlarged cross-section of the distal end 41 of the flexible tubular member 110 at 3F in FIG. 3D. The distal fill port 332 is shown positioned from 0.25 mm to 0.45 mm from the distal end of the flexible tubular member 110, in a specific example 0.354 mm from the distal end. The distal fill port 332 is in fluid communication with the first fluid channel 316. FIG. 3F is a cross-section of the flexible tubular member 110 with the first fluid channel 316 and the second channel 318 taken at 3F-3F in FIG. 3D. As shown in FIG. 3G, the proximal fill ports 322, 324, 326 are positioned at a location closest to the proximal end of the dilation balloon 150 engaging the flexible tubular member 110.

FIG. 3H illustrates a cervical dilation device 100 with an external pressure control device 350 engaging the proximal end of the cervical dilation device 100. The pressure control device 350 can be provided with any of the disclosed dilation devices without departing from the scope of the disclosure. The external pressure control device 350 has a housing 351 and is in fluid communication with the cervical dilation device 100 via, for example, a tubular connector 352. The pressure control device 350 can also operate as a pressure reservoir by incorporating a reservoir within the pressure control device housing or external to the housing (as shown by external reservoir 370 in dashed lines connected to the pressure control device 350 via a fluid delivery tubing 371). In some examples, the pressure control device 350 is an external pump that can be wearable. In another example, as shown, an on/off button 354 (e.g., operational controller) is provided which is activatable from an external surface of the housing 351. The pressure control device 350 or pump can have three buttons 356, 357, 357 to, for example, allow a user or provider to select a pressure (e.g., low pressure, medium pressure, high pressure). In other configurations, two buttons (e.g., up/down pressure change controller) can be provided which allow a user or provider to select increase pressure or decrease pressure. In still another example, a pressure control slider or pressure control knob can be provided to increase the pressure when the slider is advanced in a first direction and decrease the pressure when the slider is moved in an opposite direction. Interfaces other than buttons and sliders can be used without parting from the scope of the disclosure.

As will be appreciated by those skilled in the art, there are many advantages of providing pressure control options, including preset pressure control options, to deliver constant pressure. Pressure control options can be provided that take into account the age of the patient, pain tolerance, parous versus non-parous etc. For example, the three buttons 356, 357, 358 shown in FIG. 3H can be configured to allow the provider or user to make a selection such as low pressure, medium pressure and high pressure to accommodate the variability in pain tolerance of a patient.

The pressure control device or pump could be operable in conjunction with a custom algorithm pre-programed into a logic device which is operable to maximize efficacy while minimizing discomfort to the patient. The logic device is operable to provide logic or processing functions such as logic control. Logic devices include, by way of example and not limitation, microprocessors, digital signal processors (DSPs), application specific integrated circuits (ASICs), systems-on-chip (SoCs), programmable logic devices (PLDs), programmable logic arrays (PLAs), field programmable grid arrays (FPGAs), microcontrollers and analog and mixed-signal devices. The logic control can include, for example, one or more of a linear ramp up in pressure (e.g., change of pressure proportional to input), a sawtooth waveform increase of pressure, a sinusoidal waveform increase in pressure, a stair step-up in pressure, etc. A pressure relief feature can also be provided that is patient accessible should the patient discomfort become too great.

Where an external pressure control device 350 controls the cervical dilation device 100, the pressure control device 350 can be further operable to control the amount of fluid in the cervical dilation device 100 and can include user input, a measurement of input, a processing logic, and a memory. As discussed above, the logic control can generate a control signal to control the delivery of fluid to the cervical dilation device 100. Control signals can be generated that control the amount of fluid deliverable by the external pressure control device 350.

The pressure control device can be operable to deliver a pre-selected fixed pressure (low, medium, or high) in either a fixed or pulsed fashion. For example, a younger woman who has not had a prior vaginal birth delivery may be best suited for a low pressure pulsed pressure delivery algorithm such as 2psi for 20 seconds followed by 0.5psi for 40 seconds and so on.

Additionally, the volume of fluid (air or sterile saline for instance) can be monitored via a display on a controller such that the provider can know approximately at what diameter the cervix has been dilated at any time during the procedure. The display can be incorporated in the housing of the external pressure control device 350.

An audible feedback feature can also be included in communication with the controller unit such that if any pressure is detected outside of the predicted range a warning sound can be emitted. Audio feedback can also be provided at various predicted dilation diameters based on the volume of fluid delivered throughout the procedure including a final audio signal indicating target dilation has been achieved. A speaker delivering the audible feedback can be incorporated into the external pressure control device 350.

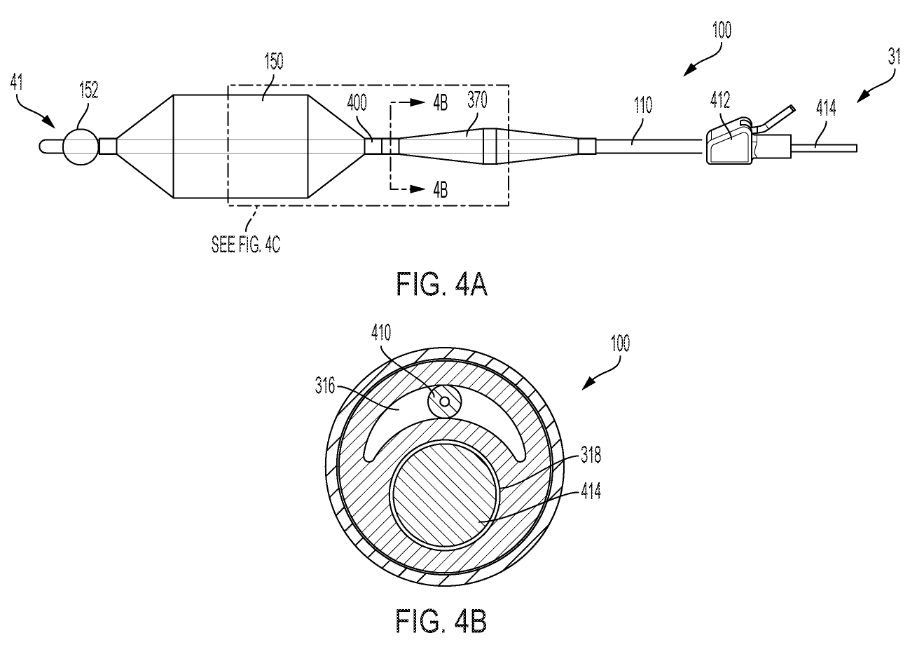

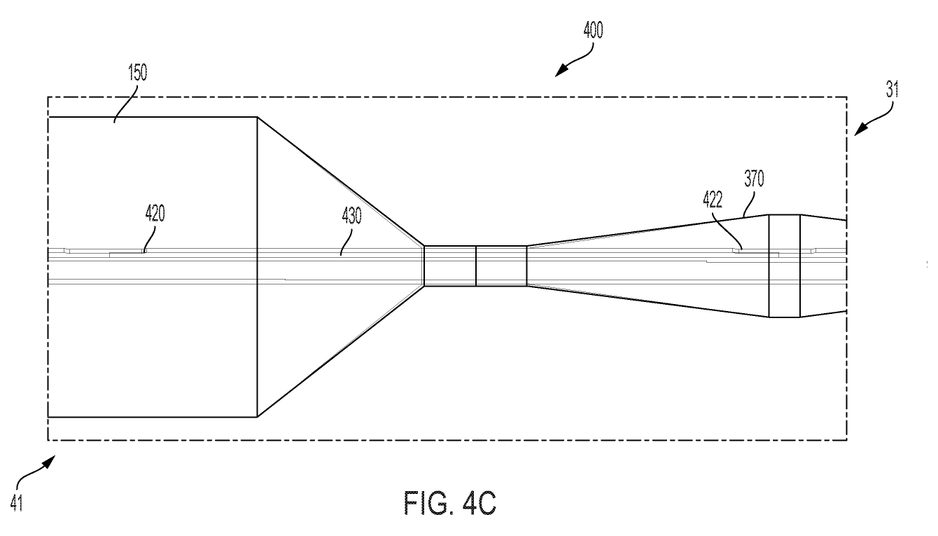

FIGS. 4A-C illustrates another exemplar cervical dilation device 100 with a flow restrictor 400 incorporated therein. A distal balloon 152 is provided towards or at the distal end 41 of the elongated flexible tubular member 110. The cervical dilation device 100 is shown with a Y connector 412 at a proximal end 31 with a stylet 414 positioned therein. The stylet is typically a thin, stiff wire or rod that provides rigidity and pushability to facilitate advancement of the inflation device. The stylet can provide additional structural support to the inner shaft during pleated folding of the dilation balloon 150. The Y connector 412 has three parts: a Y tube adapter, a straight adapter and an elbow adapter.

The flow restrictor 400 can be incorporated between an external reservoir 370 and the dilation balloon 150. The external reservoir 370, can also be a balloon. Providing a flow restrictor 400 between the external reservoir 370 and the dilation balloon 150 is operable to prevent an initial water pressure in the external reservoir 370 from being delivered to the dilation balloon 150 too quickly. As will be appreciated by those skilled in the art, fast dilation of the dilation balloon 150 could result in discomfort for the patient. By providing a flow restrictor 400 the rate at which the dilation balloon 150 is expanded can be controlled and more quickly stopped during a procedure.

In use, without a flow restrictor and using only sterile water as the fluid medium, the pressure developed in the external reservoir 370 will equalize with the pressure of the dilation balloon 150 almost instantly. When a higher viscosity fluid is used (e.g. a contrast medium mixed with sterile water) and a flow restrictor is positioned between the external reservoir 370 and the dilation balloon 150, the pressure can be slowly developed inside the dilation balloon. A force measurement in the radial direction of the balloon has shown that a target rate of 10 minutes per millimeter of balloon diameter expansion at a set force of 30N will achieve a 30mm dilation within 4 hours. The selection of 30N is an example and other forces can be used without departing from the scope of the disclosure. The selection of 30N was seen as a reasonable force based on measurement of other osmotic dilation products such as Dilapan-S® (Medicem Technologies, s.r.o.) within a similar balloon configuration.

FIG. 4B is a cross-section of the cervical dilation device 100 taken along the lines 4B-4B of FIG. 4A. As shown in FIG. 4B fluid channels 316, 318 are provided. The fluid channels 316, 318 traverse substantially the entire length of the elongated flexible tubular member 110. A first fluid channel 316 has a crescent shape that can be potted with adhesive (e.g., encapsulating the space with adhesive) to create flow restriction lumen 410 within the first fluid channel 316. The crescent shape first fluid channel 316 is a curved shape that tapers at two ends. The first fluid channel 316 can be positioned within the tubular member 110 so that the first fluid channel 316 curves about a portion of the second channel 318. The second channel 318 receives the stylet 414.

FIG. 4C is an exploded view of the portion of the cervical dilation device 100 in the circle identified by 4C in FIG. 4A. As shown in FIG. 4C, the flow restrictor 400 is part of a restrictor tubing 402 which is positioned between an external reservoir 370 and the dilation balloon 150. A first skive 420 is shown positioned within the dilation balloon 150, and a second skive 422 is shown positioned within the external reservoir 370. The first and second skive are areas where the outer cover of the tubing is removed. Skiving may be used to create tapered or stepped profiles, with dimensions of, for example 2 millimeters in length and 0.5 millimeters in width, with a tolerance of plus or minus 1 millimeter.

Restrictor tubing 430 is positioned about the elongated flexible tubular member 110 between the first skive 420 and the second skive 422. Once the cervical dilation device 100 is positioned the stylet 414 can be removed. The second channel 318 receives the stylet 414 can then be used to deliver fluid to the distal balloon 152. The skived sections 420, 422 of tubing allows fluid to travel to the external reservoir 370 and dilation balloon 150 via the first fluid channel 316.

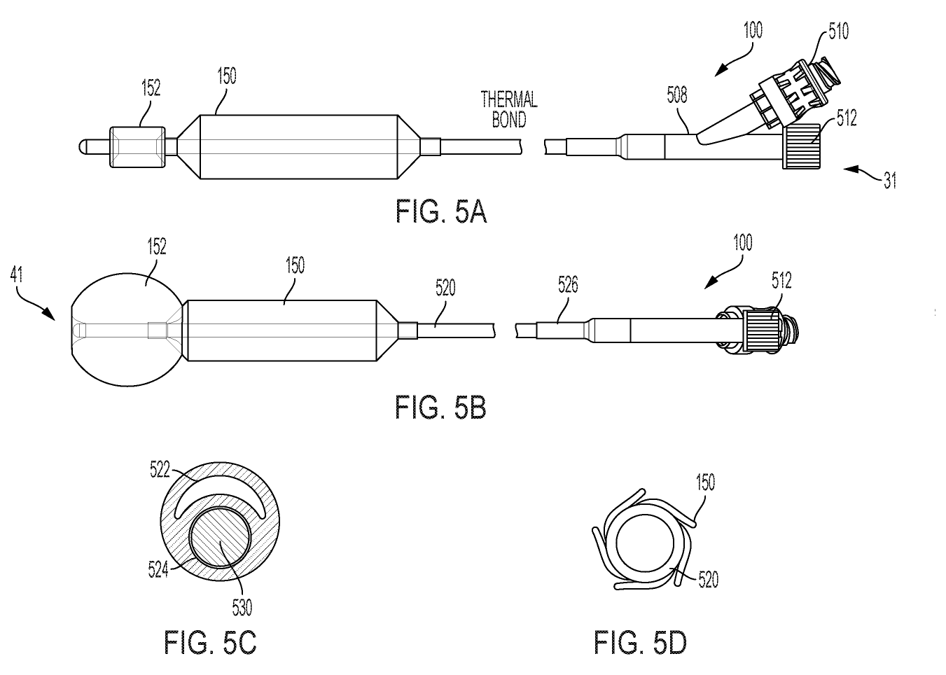

FIGS. 5A-D illustrate another exemplar cervical dilation device 100. The cervical dilation device 100 has a dilation balloon 150 and a distal balloon 152. The proximal end has a y-connector 508 with a needleless injection site 510 adjacent a male Luer lock cap 512 on one side and a multi-lumen tubing 520 on the other side. The needleless injection site 510 is operable to inflate the distal balloon 152 and the dilation balloon 150. A heat shrink material 526, such as polyolefin resin, is provided distal to the y-connector to provide strain relief for the cervical dilation device. As shown in FIG. 5A, the dilation balloon 150 has a tapered proximal end and a tapered distal end. The distal end of the cervical dilation device 100 is sealed thermally or with a suitable glue. The inflated distal balloon 152 will roll over to remove any gap between the tapered distal end of the distal balloon 152 and the dilation balloon 150 as shown in FIG. 5B.

FIG. 5C illustrates a cross-section of the multi-lumen tubing 520 with a dilation inflation lumen 522, and an anchor inflation lumen 524. A stylet 530 is positioned in the anchor inflation lumen 524. FIG. 5D illustrates a cross-section of the dilation balloon 150 surrounding the multi-lumen tubing 520 wherein the dilation balloon 150 has a pleat folded profile prior to inflation.

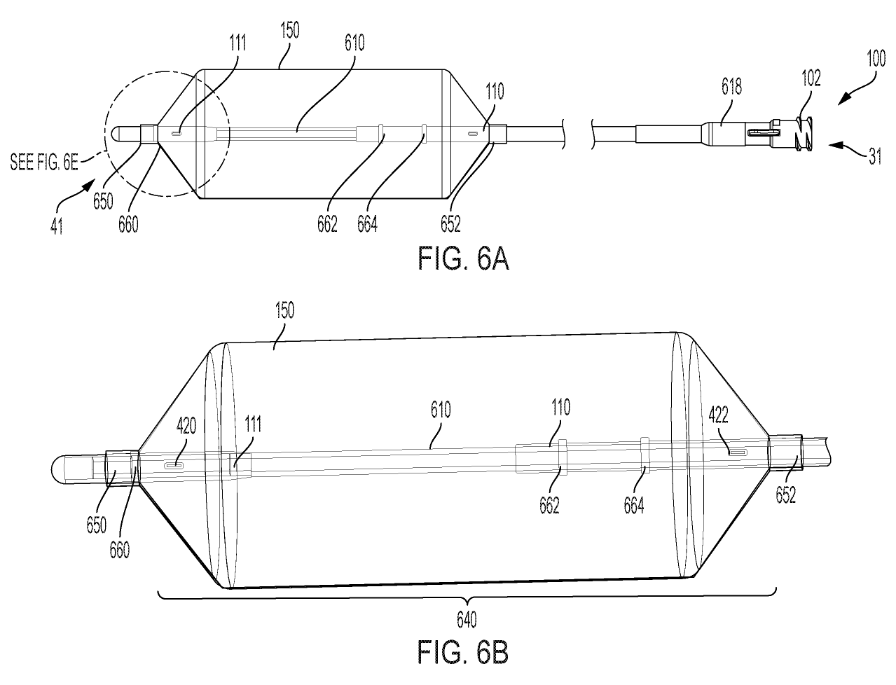

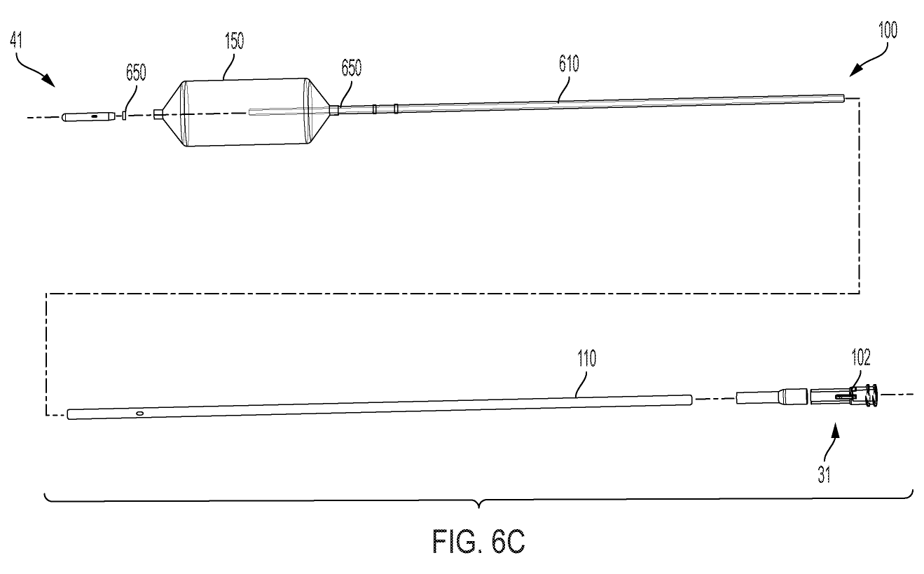

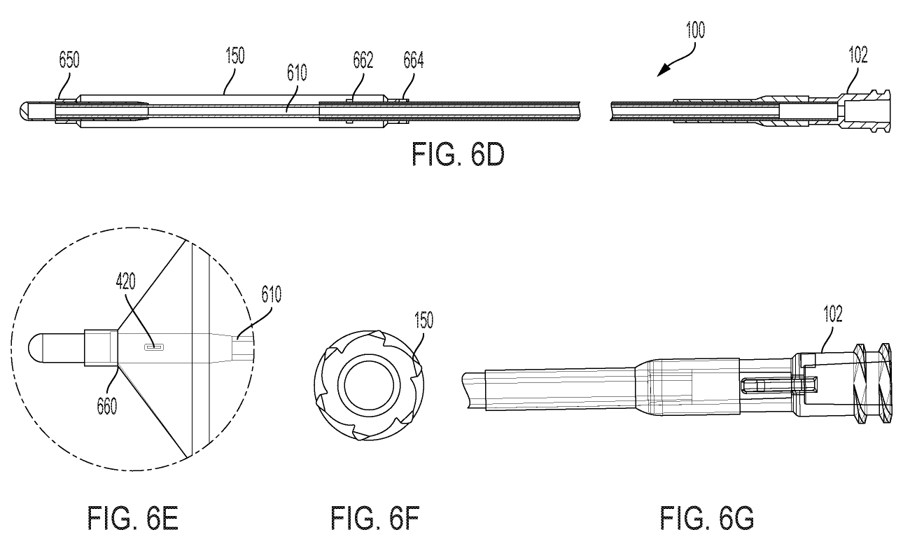

FIGS. 6A-6G illustrates a cervical dilation device 100 with a telescoping inner shaft 610 positioned within an outer tubular member 110. The sheath and stylet are not illustrated. The cervical dilation device 100 comprises: a dilation balloon 150 with a proximal dilation balloon end and a distal dilation balloon end, the inflatable dilation balloon is operable to expand from an unexpanded configuration to an expanded configuration with an external inflatable dilation balloon diameter up to 30mm; a first outer tubular member 110 connected to the dilation balloon 150 at the proximal dilation balloon end and a second outer tubular member 111 connected to the dilation balloon 150 at the distal dilation balloon end; a telescoping inner shaft 610 positioned within the first outer tubular member 110 and telescopically extendable to the distally positioned second out shaft tubing 111, the telescoping inner shaft 610 operable to prevent kinking in response to tension applied to the cervical dilation device when the dilation balloon 150 is inflated; tubing thermally bonded to the outer shaft and one or more skives 420, 422 positioned on the outer tubular member 110, 111; and one or more radiopaque marker bands 660, 662, 664 positioned on the outer tubular member 110, 111, the one or more marker bands operable to provide dimensional reference during operation of the cervical dilation device 100. In contrast to the stylet, the telescoping inner shaft is a multi-component sliding shaft system that includes nested cylindrical sections that can extend or retract axially. The telescoping functionality of the telescoping inner shaft 610 allows adjustment of the length and/or precise advancement and retraction of the dilation balloon 150 by allowing the components of the telescoping inner shaft 610 to slide relative to one another. The telescoping inner shaft 610 also facilitates fine control of movement of the cervical dilation device 100 while preventing kinking during use.

The distal balloon bond and the proximal balloon bond keep the telescoping components at a desired length. The distal balloon bond is formed on the short distal outer section and is fixed to the distal end of the inner telescoping tubing. The proximal end of the inner telescoping tubing floats freely within the proximal outer tubing. The proximal balloon bond is formed on the distal end of the long piece of the outer tubing. The proximal end of the long outer tubing is where the Luer connector is bonded. This configuration enables the device to operate without stops because the balloon itself determines the amount of movement of the telescoping tubing as it moves within the outer tubing. A small amount of annular clearance is provided between the inner tubing and the outer tubing to allow for movement of the telescoping tubing and passage of fluid for inflating and deflating the ballon. The fluid also provides additional lubrication between the tubing elements. The inner tube and outer tube can be formed from any suitable material, such as material that minimizes friction. Suitable material includes, but is not limited to polyethylene (PE), polyurethane (PU), and nylon.

FIG. 6A illustrates a side view of an assembled cervical dilation device 100. The cervical dilation device 100 is sized and constructed for cervical dilation. The dilation balloon 150 is folded (pleated) prior to deployment into its deployed state (see FIG. 6F). The dilation balloon 150 may be constructed from a polyamide material, which may provide high mechanical strength and flexibility, ensuring reliable performance under demanding conditions. Suitable expandable material includes VESTAMID® ML21 from Evonik Industries AG. The dilation balloon 150 defines a dilation portion 640 of the cervical dilation device 100. The dilation balloon 150 length may have a tolerance of plus or minus 2.5 millimeters, while the tubing dimensions may have tolerances of plus or minus 0.005 inches. Tailored dimensions may also be specified for other components, such as an outer shaft length of 30 millimeters with a tolerance of plus or minus 1.5 millimeters, an inner shaft length of 25 millimeters with a tolerance of plus or minus 2 millimeters, and a tail cut length of 10 millimeters with a tolerance of plus or minus 2 millimeters. Transition geometries may be chamfered or rounded to reduce stress concentrations, further improving durability and performance of the device.

The dilation balloon 150 can be made of a tough, non-compliant material such as nylon which increases its tolerance of high pressures. The main fluid channel can be accessed through the Luer, then through the outer tube and then some fluid can also pass between the annular space of the inner tube and outer tube to the balloon. A shutoff valve can be included in some configurations or as an accessory.

The dimensions of the dilation balloon 150 may include a diameter of 30 millimeters and a length of 57.5 millimeters, with a tolerance of plus or minus 2.5 millimeters to maintain consistency across manufacturing. The dilation balloon 150 may be capable of withstanding inflation pressures up to 4 atmospheres, with a nominal operating pressure of 1 atmosphere.

Pressure can be monitored by the user. Alternatively, a separate pressure gauge can be provided, or the user can target a particular volume provided in the instructions for use.

In an inflated state (deployed condition), the dilation balloon 150 can have, for example, a length of approximately 57.5mm (2.264in) with a diameter of approximately 30mm (1.181in), allowing for effective dilation at the set nominal pressure of 1 ATM. The distal end of the dilation balloon 150 and the proximal end of the dilation balloon 150 are shown terminated by thermal bonds 650, 652 and UV adhesive joints, which affix the dilation balloon 150 to the outer tubular member 110 and may secure the marker bands to the outer tubular member 110. The outer tubular member 110, 111 may be available in various dimensions, such as an inner diameter of 0.105 inches and an outer diameter of 0.135 inches for one type, and an inner diameter of 0.138 inches with a width of 1 millimeter for another type. Specific colorants may be incorporated into the tubing to improve visibility and identification during medical procedures. The tubing may be designed to integrate seamlessly with other components, ensuring a robust and leak-proof assembly that may meet the stringent requirements of medical-grade devices. Additionally, the outer diameter of the tubing may be specified as 0.100 inches with a tolerance of plus or minus 0.005 inches, while the inner diameter may be specified as 0.060 inches with a tolerance of plus or minus 0.005 inches, ensuring consistent performance. The thermal bond can be configured to secure tubing to the outer shaft. The thermal bonds may also prevent separation in response to axial forces applied to the outer shaft when the dilation balloon is expanded.

The telescoping inner shaft 610 can be formed from of PEBAX 55D, and have, for example, an inner diameter (ID) of 0.105in and outer diameter (OD) of 0.135in. The telescoping inner shaft 610 can extend from the proximal end to the distal end and through the dilation balloon 150, and is operable to provide axial support and a conduit for inflation. Additionally, the telescoping inner shaft 610 is operable to prevent buckling or kinking of the cervical dilation device 100 and dilation balloon 150 during inflation. The telescoping inner shaft 610 overcomes challenges by maintaining structural integrity and functionality during use. Kinking under tension as pressure is applied to a dilation balloon 150 during inflation can lead to compromised performance and potential procedural complications. Providing the telescoping inner shaft 610 reduces or eliminates the tendency of the cervical dilation device 100 to kink or buckle.

The outer tubular member 110 includes skived features distally and proximally within the dilation balloon 150 facilitate secure dilation balloon bonding, with each skive 420, 422 extending, for example, along a 2mm length and a 0.5mm width. One or more skive ports can be made by mechanical cutting, laser cutting, or by application of a heat source. The skive ports can be located anywhere within the inner tubing withing the balloon section. In at least some configurations, two skive ports are positioned at the distal end and two skive ports are positioned at the proximal end of the inner tubing within the balloon.

The telescoping inner shaft 610 and the outer tubular member 110, when assembled, create the functional working length of the cervical dilation device 100. A suitable working length is, for example, 251.10mm. The outer tubular member 110, can also be composed of 55D PEBAX, and have an inner diameter of, for example, 0.138in, a 1mm width. The outer tubular member 110 is also operable to contribute to the telescoping construction for controlled flexibility if the cervical dilation device 100. The assembled telescoping inner shaft 610 maintains a required minimum bend radius of 5in to ensure safety and ease of manipulation during use.

One or more marker bands 660, 662, 664 are provided formed of 304 stainless steel, with, for example, a 0.137in ID, 1mm width, and 0.002in wall thickness, is embedded within the outer tubular member distal section to enable radiographic visualization during placement of the cervical dilation device 100. The marker bands 660, 662, 664 can be over-molded, bonded, or interference-fit. The radiopaque properties of the marker bands may ensure that the cervical dilation device can be effectively monitored and guided, contributing to the overall accuracy and reliability of the cervical dilation device. The dimensions of the marker bands may be controlled to ensure proper fit and functionality, aligning with other components of the cervical dilation device to maintain structural integrity. The marker bands provide for better positioning and visualization when the device is deployed.

A fluid fitting 102, such as a female Luer connector, is mounted on the proximal shaft end for attachment to inflation media. Heat shrink tubing 618, can, for example, be made from MT2000 medical-grade polyolefin, ensures a secure and sealed junction. MT2000 is a thin wall polyolefin heat shrink tubing available from, for example, PMG Company.

The adhesive bonds can utilize UV-cured urethane acrylate compositions.

In at least some configurations, the maximum folded/pleated balloon exterior diameter shown in FIG. 6D does not exceed 6.00mm (0.236in). The folded/pleated balloon can be housed within a balloon protector or packaging sheath prior to deployment of the cervical dilation device 100. Thus, for example, completing an assembly for a cervical dilation device 100, a packaging sheath of, for example, HDPE with an inner diameter of 0.242in can be provided that shields the folded dilation balloon 150 and cervical dilation device 100 during shipment and handling, maintaining sterility and protecting the low-profile pleated balloon until use.

The cervical dilation device 100 is supplied in a pre-deployment configuration where the dilation balloon 150 is pleated and folded with a balloon protector sheath and an internal stylet to facilitate straightening and insertion. As persons skilled in the art will appreciate, the balloon protector sheath is a tubular covering that either goes over or works in conjunction with the balloon to, for example, protect the balloon during introduction and withdrawal by reducing friction and risk of balloon damage. Chamfered or rounded geometries are implemented wherever transition zones occur to minimize patient trauma and enhance device integrity.

### Systems

The disclosed cervical dilation devices with a balloon and an elongated flexible tubular member, such as a catheter, is part of a system for cervical dilation or cervical compression. The system includes a cervical dilation device and a reaction solution having a target reaction rate resulting a target volumetric expansion operable to give off a gas as one or more products of the reaction. The volumetric expansion resulting from the reaction is operable to cause the dilation balloon to increase in volume (e.g., expand).

### Methods

### A. Pre-birth Cervical Dilation

The cervical dilation device 100 is advanced through the cervical canal into the uterus. Once the distal end of the cervical dilation device 100 is positioned in the uterus 30, the distal balloon 152 is expanded. In a distal balloon is present, the distal balloon 152 can be dilated with a suitable material, such as a sterile saline solution, sterile water or air. Dilation of any distal balloon can occur quickly to position the cervical dilation device 100. Where a telescoping inner shaft 610 is provided the telescoping inner shaft 610 can be advanced through the cervical dilation device 100 prior to inflation of the dilation balloon 150.

Once the distal balloon 152, if present, is dilated, a liquid solution is delivered to the dilation balloon 150 to begin slow expansion of the dilation balloon 150. The liquid solution will contain materials that react at a known rate and give off a gas as one or more products of the reaction. The gas results in expansion of the dilation balloon 150. The quantity and ratios of the materials provided can be selected to provide a controlled and predictable dilation rate of the dilation balloon 150, dilation time and final pressure. The liquid solution can be liquid or combinations of solid and liquid capable of achieving a liquid to gas reaction resulting in expansion. The ability to use a fixed volume to produce predictable pressure results in a mechanical change to the surrounding tissue over several hours.

A chemical reaction such as a fermentation reaction is a suitable reaction for achieving expansion. As will be appreciated by those skilled in the art, when a yeast cell consumes sugar, ethanol and carbon dioxide gas are produced. The phase change between liquid and gas produces volumetric expansion and the volume of yeast, sugar and water is less than the volume of the ethanol and carbon dioxide gas. Expansion using a fermentation reaction also allows for the use of food grade, non-toxic materials. The fermentation process can combine one or more of: dry yeast (0.5 tsp – 1.5 tsp), sucrose (0 tsp – 3 tsp), and water (30 mL – 50 mL at a temperature of from 100-130°F). The yeast can be, for example, Fleischmann’s instant dry yeast, fast acting, gluten free, no preservatives. The sucrose can be a science reagent grade sucrose. The expansion is allowed to occur over 3 to 5 hours, or in a specific example 4 hours. In another expansion scenario the expansion will occur over 10 to 14 hours, or preferably 12 hours. The cervical dilation device does not rely on osmosis for fluid. Availability of body fluid can vary from patient to patient. Therefore, providing a measured fluid provides a predictable dilation. Other reactions that produce a safe gas product, such as CO2, can be used as well to achieve expansion of the balloon under this method. In some implementations, a rate limiting membrane can be provided to control or slow the rate of reaction. In other configurations, a hydrogel can be used to provide controlled or slow expansion of the cervical dilation device.

Additionally, the water to be injected into the reservoir balloon can be mixed with a contrast medium that is operable to increase the viscosity of the mixture. The contrast media can be the same media that is currently used directly in the bloodstream for better contrast in radiological procedures (e.g., gadolinium or iodinated contrast). Using a contrast already used in radiological procedures provides additional safety, In the event of a balloon failure (e.g., rupture or leak), the fluid material it is still safe for the patient. An additional benefit of including a contrast media is that the increased viscosity is operable to further slow the rate that the dilation balloon expands and provides pressure to the tissue. Slowing, or better controlling the rate of dilation, improves patient tolerance to the procedure and makes dilation of the cervix more tolerable.

Additionally, the patient can use the pressure controller to increase or decrease the amount of pressure applied by the dilation device.

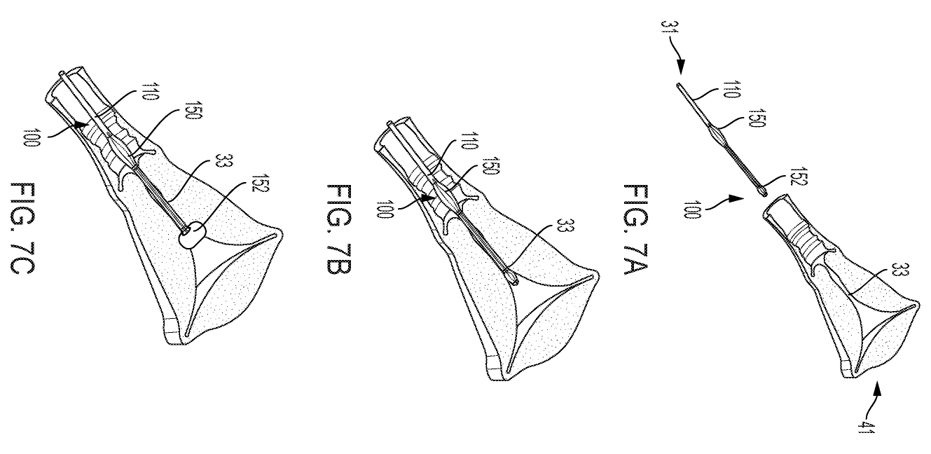

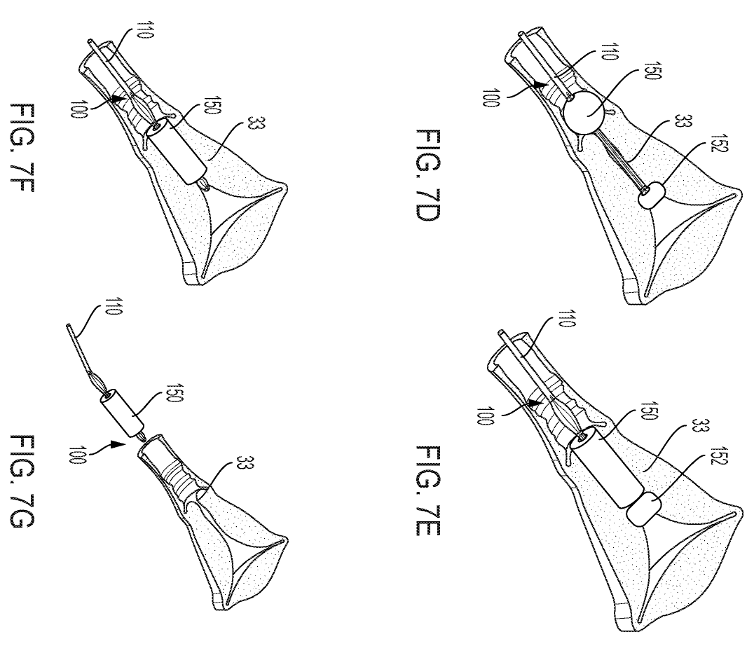

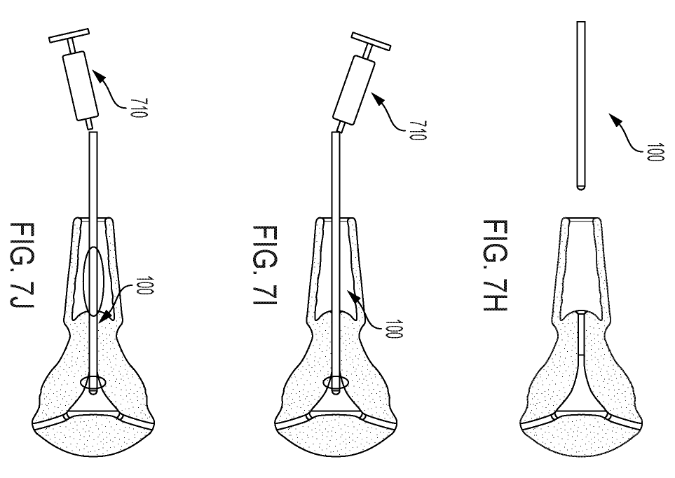

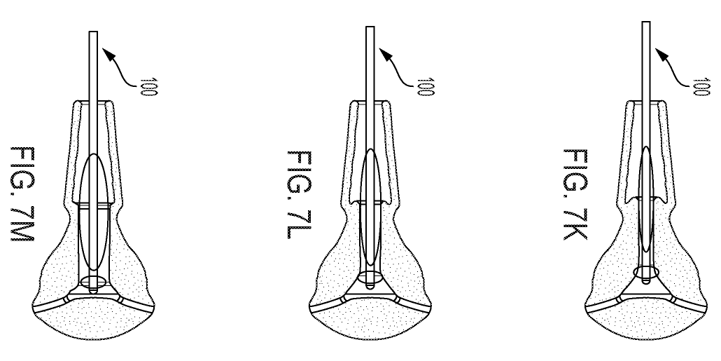

The method is illustrated more particularly in FIGS. 7A-G. In FIG. 7A, the cervical dilation device 100 is shown ready for insertion. FIG. 7B the cervical dilation device 100 has been passed through the cervical canal 33. FIG. 7C cervical dilation device 100 is in position and the distal balloon 152 has been dilated. In FIG. 7D the dilation balloon 150 has started to expand In FIG. 7E the dilation balloon 150 has begun expanding in the cervical canal 33. In FIG. 7F the distal balloon 152 has been deflated and the diameter is reduced from the anchor diameter. In FIG. 7G, the dilation balloon 150 has been removed.

Because the expansion relies of the dilation balloon 150 on a slow reaction, the procedure can be easily be stopped any time prior to the final target dilation. Any generated CO2 can be removed to reduce the balloon diameter to allow for easy removal of the cervical dilation device 100.

FIGS. 7H-M illustrate a process of operating and inflating the cervical dilation device. In FIG. 7H the cervical dilation device 100 is about to be inserted. FIG. 7I shows the cervical dilation device 100 inserted and the distal balloon 152 inflated. FIG. 7J shows delivery of a pre-mixed media into the dilation balloon 150. The pre-mixed media, liquid, fills a proximal segment of the balloon since there is less resistance in the proximal segment of the balloon than the portion of the balloon positioned within the cervix. In FIGS. 7K-M the media reaction continues to generate a harmless gas, such as CO2, within the dilation balloon which begins causing dilation within the cervix. As appreciated, by those skilled in the art, the dilation can continue, for example, up to 4 hours or more and can be stopped at any time, or when target dilation is achieved, by releasing gas product by allowing the gas to be released through an external valve or via a syringe. Other reactions that convert a liquid to a gas can also be utilized such as the combination of a lower pH material such citric acid and a higher pH material such as baking soda. These materials tend to react quickly so a permeable membrane can be used to help control the rate of the reaction. Many of these non-toxic, readily available materials exist and could be utilized to create volume within a compliant or non-compliant material.

For some methods, a patient or practitioner can control the inflation of the cervical dilation device via an external pressure control device. Control can include, for example, selecting between preset pressure levels (e.g., high, medium, low) or releasing pressure. Additionally, the rate of inflation can be impacted by the inclusion of a flow restrictor as shown in FIG. 4.

### B. Compression to Address PPH

The cervical dilation device 100 is advanced through the cervical canal 33 into the uterus 30. Once the distal end of the cervical dilation device 100 is positioned in the uterus, the dilation balloon 150 is expanded. The dilation balloon can be dilated with a suitable material, such as a sterile saline solution, sterile water or air. Dilation can occur quickly to provide compression on the uterus to reduce or eliminate blood loss.

The cervical dilation device 100 can be the same cervical dilation device used for dilation when the dilator is removed and kept in a sterile field. In this application, the cervical dilation device is used to first dilate the cervix, then removed and kept in a sterile field. After the vaginal birth is completed, if bleeding is noted, the cervical dilation device 100 is re-inserted with both balloons in the uterus or just the distal balloon in the uterus and the dilation balloon in the cervix. The cervical dilation device is expanded to apply compression needed to address PPH. This process allows compression to be applied at a size significantly less than 500 mL.

The cervical dilation device 100 can optionally include a compound or composition, such as a pharmacologic or drug compound or composition, capable of delivering a topical agent, such as a hemostatic agent (whether compound or composition), to the cervical and/or intrauterine tissues to assist in the management of postpartum hemorrhage. As will be appreciated by those skilled in the art, the hemostatic agent can be a drug, biologic or a device. For example, systemic hemostatics such as prothrombin complex concentrates or recombinant clotting factors are treated as drugs, whereas many topical hemostatic dressings (e.g., chitosan or plant‑starch dressings) are regulated as medical devices rather than drugs, even though they promote hemostasis.

At least a portion of the outer surface of the dilation member of the cervical dilation device 100 is coated, impregnated, or otherwise associated with the compound or composition. The compound or composition can also be provided via a sleeve that fits over the dilation member of the cervical dilation device 100. Suitable compounds or compositions comprise an antifibrinolytic agent, a uterotonic agent, a procoagulant agent, or a combination thereof, formulated for local delivery upon contact with blood or other body fluids. The compound or composition can be selected, for example, to include tranexamic acid or another topical hemostatic compound that, when released from the surface of the cervical dilation device 100 during or after cervical dilation, promotes clot stability and reduces ongoing bleeding, while the underlying mechanical expansion and/or tamponade provided by the cervical dilation device 100 contributes additional hemostatic effect. In some implementations, the compound or composition is carried in or on a polymeric matrix, porous layer, gauze, or other substrate disposed on the dilation element of the cervical dilation device 100 so that release of the compound or composition is localized to the cervix and lower uterine segment, thereby reducing the need for systemic administration and providing targeted adjunctive therapy for postpartum hemorrhage.

### KITS

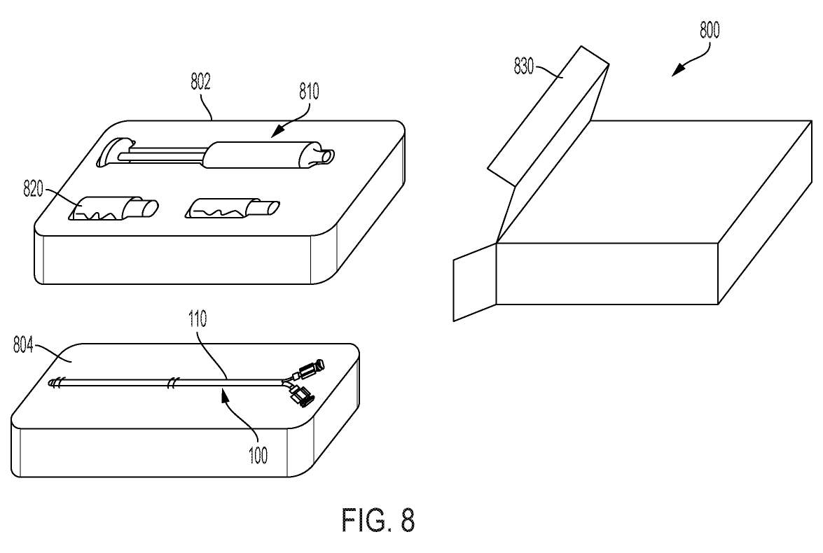

FIG. 8 illustrates a kit 800 for a cervical dilation device 100. The contents of the kit 800 can be provided in a box 830. A tray 802 can be provided which holds one or more syringes 810 and one or more vials 820 containing materials for use in the inflation process as described herein. The cervical dilation device 100 can be provided in a suitable flexible, breathable, water and bacterial resistant peel pouch. A suitable pouch can be made from Tyvek® available from DuPont. A sleeve can be provided that has a compound or composition. A packaging sheath can be positioned around the inflatable balloon to protect the inflatable balloon during storage and transport.

### Manufacturing

The cervical dilation devices disclosed herein can be manufactured using medical‑grade polymeric tubing, balloon films, adhesives, and heat‑shrink materials selected to provide dimensional stability, kink resistance, and biocompatibility. All or some of the materials can be impregnated with or coated with a compound or composition.

In at least some configurations, the outer tubular member 110 and/or telescoping inner shaft 610 are formed from PEBAX 55D or similar thermoplastic elastomer, extruded to provide specified inner and outer diameters with tolerances of approximately plus or minus 0.005 inches to maintain consistent flow characteristics and mechanical performance. In some examples, the outer tubular member has an inner diameter of about 0.100 inches and an outer diameter of about 0.138 inches, while the telescoping inner shaft has an inner diameter of about 0.105 inches and an outer diameter of about 0.135 inches, thereby defining a controlled annular clearance for fluid flow and telescoping movement.

The inflatable dilation balloon 150 can be fabricated from a non‑compliant or semi‑compliant polyamide film, such as a nylon material or VESTAMID® ML21, using blow‑molding or similar balloon‑forming processes to yield an expanded outer diameter of up to about 30 mm and a length of about 57.5 mm, with a tolerance of plus or minus 2.5 mm. The balloon material is selected to withstand inflation pressures up to about 4 atmospheres and to provide a nominal operating pressure of approximately 1 atmosphere without substantial radial growth beyond the target diameter. After forming, the balloon is trimmed to length and, in some configurations, pleated or folded to achieve a maximum folded outer profile not exceeding about 6.0 mm, thereby facilitating passage through the cervical canal.

Assembly can proceed by first preparing the outer tubular member 110 and the telescoping inner shaft 610 to their target lengths. For example, an outer shaft length of about 30 mm with a tolerance of plus or minus 1.5 mm, an inner shaft length of about 25 mm with a tolerance of plus or minus 2 mm, and a tail cut length of about 10 mm with a tolerance of plus or minus 2 mm can be specified for a particular configuration. Skive features 420, 422 may be formed in the outer tubular member in locations corresponding to the distal and proximal ends of the dilation balloon by mechanical cutting, laser cutting, or controlled thermal processing, with each skive extending, for example, about 2 mm in length and about 0.5 mm in width.

The dilation balloon 150 is then bonded to the outer tubular member 110 and the distal outer member 111 at selected balloon bond regions. Thermal bonding can be used to fuse the balloon ends 650, 652 to the underlying tubing, and UV‑curable urethane acrylate adhesive may be applied to reinforce the joints and secure one or more marker bands 660, 662, 664 positioned beneath or adjacent the balloon. Marker bands formed from 304 stainless steel, or other radiopaque materials, can be manufactured with an inner diameter of about 0.137 inches, a width of about 1 mm, and a wall thickness of about 0.002 inches, and then interference‑fit, over‑molded, or adhesively bonded onto the outer tubular member prior to balloon bonding. The distal balloon 152, when present, can be formed from a compliant silicone or polyurethane material and bonded over a distal section of the tubing using similar thermal or adhesive bonding techniques to define a distal anchor balloon region.

Multi‑lumen tubing 520, where used, can be extruded to provide separate lumens 522, 524 for dilation and anchor inflation and, in some embodiments, for temporary stylet placement. Dimensions of the lumens and ports are selected to provide desired flow characteristics and restrictor performance; for example, channel diameters may range from approximately 0.02 mm to 1.0 mm, with specific examples including a first channel diameter of about 0.039 mm and a second channel diameter of about 0.079 mm. Distal and proximal fill ports 332, 322, 324, 326 can be formed by drilling, laser ablation, or punch‑cutting through the tubing wall, with port diameters between about 1.0 mm and 2.0 mm and port spacing of about 5 mm to 15 mm, such as 10 mm.

In configurations incorporating a flow restrictor 400, a portion of a crescent‑shaped first channel 316 can be potted with an adhesive to form a narrowed flow restriction lumen 410, or a length of restrictor tubing 402, 430 with reduced inner diameter can be inserted between an external reservoir 370 and the dilation balloon 150. Skived sections 420, 422 in the tubing are positioned so that fluid communication is provided between the restrictor tubing and the balloon and/or reservoir, while maintaining structural integrity of the outer tubular member. Adhesive potting materials and restrictor tubing dimensions are selected to achieve a target inflation rate, such as on the order of several minutes per millimeter of balloon diameter expansion at a specified radial force.

The telescoping inner shaft 610 may be fabricated as a nested tube assembly, with an inner tube and outer tube formed from low‑friction materials such as polyethylene, polyurethane, nylon, or PEBAX 55D, sized to maintain a small annular clearance that allows both axial sliding and passage of inflation fluid. The distal end of the inner telescoping tube is bonded to a distal outer shaft segment at or near the distal balloon bond, while the proximal end of the inner tube is free to move within the proximal outer shaft segment, allowing the balloon expansion to dictate the extent of telescoping travel. Thermal bonds and UV‑cured adhesive joints can be used at these interfaces to withstand axial tensile loads encountered when the balloon is tensioned against the cervix or uterus without detachment or leakage.

Proximal connectors such as Luer or Y‑connectors 102, 412, 508 can be molded from medical‑grade polymers and bonded to the proximal ends of the tubing using solvent bonding, thermal bonding, and/or UV‑curable adhesives. Heat‑shrink tubing 618, such as MT2000 medical‑grade polyolefin, can be applied over junction regions between the tubing and the connectors to provide strain relief and a fluid‑tight seal. In some configurations, a needleless injection site 510 and male Luer lock 512 are integrated into the proximal assembly, allowing controlled access to the inflation lumens.

After assembly, the dilation balloon 150 is pleated or otherwise folded into a low‑profile configuration and a balloon protector or packaging sheath, such as an HDPE sheath with an inner diameter of about 0.242 inches, is placed over the balloon and distal shaft portion. The completed device, optionally with a removable stylet positioned in an anchor lumen or stylet lumen for added rigidity, is then packaged into a sterile pouch, such as a Tyvek®‑based peel pouch, and sterilized using an appropriate method (e.g., ethylene oxide or gamma irradiation) validated to maintain material properties and sterility assurance. Devices can be manufactured in a range of sizes and working lengths (for example, a working length of about 251.10 mm and a minimum bend radius of about 5 inches) to accommodate different patient anatomies while maintaining consistent functional performance across the product line.

A sleeve can be manufactured from any of a variety of materials and impregnated or coated with a compound or composition.

As will be appreciated by those skilled in the art, throughout this specification, unless the context requires otherwise, the word “comprise” or variations such as “comprises” or “comprising”, will be understood to imply the inclusion of a stated integer or group of integers but not the exclusion of any other integer or group of integers.

The present teachings may also extend to one or more of the following numbered clauses:

Clause 1. A cervical dilation device comprising:

an elongated flexible tubular member having a proximal end and a distal end with a fluid channel;

an inflatable dilation balloon disposed adjacent the distal end of the elongated flexible tubular member, the inflatable dilation balloon having a proximal balloon end and a distal balloon end and being configured to expand to a diameter of up to about 30 mm;

a fluid lumen extending from the proximal end toward the inflatable dilation balloon;

a telescoping inner shaft disposed within an outer shaft portion of the elongated flexible tubular member, the telescoping inner shaft configured to translate relative to the outer shaft portion and resist kinking of the inflatable dilation balloon when the dilation balloon is subjected to tension; and

a flow restrictor fluidly coupled between a proximal fluid reservoir region and the inflatable dilation balloon and configured to control a rate of fluid flow into the inflatable dilation balloon.

Clause 2. The cervical dilation device of clause 1, wherein the telescoping inner shaft comprises an inner tube and an outer tube defining an annular clearance therebetween to permit passage of inflation fluid toward the inflatable dilation balloon.

Clause 3. The cervical dilation device of clause 1, wherein the inflatable dilation balloon is formed of a non‑compliant polymeric material selected from nylon, polyamide, polyethylene, polyurethane, or combinations thereof.

Clause 4. The cervical dilation device of clause 1, wherein the inflatable dilation balloon is configured to withstand an inflation pressure of at least about 4 atmospheres.

Clause 5. The cervical dilation device of clause 1, further comprising a proximal connector configured to couple to a syringe or other fluid source to deliver inflation fluid to the inflatable dilation balloon.

Clause 6. The cervical dilation device of clause 1, wherein the elongated flexible tubular member has a curved distal portion configured to facilitate insertion through a cervical canal.

Clause 7. The cervical dilation device of clause 1, wherein the inflatable dilation balloon is configured, in a first mode of operation, to dilate a cervical canal over a period of time and, in a second mode of operation, to compress a uterus to treat postpartum hemorrhage.

Clause 8. The cervical dilation device of clause 1, wherein the flow restrictor comprises a narrowed lumen region, orifice, valve, or porous plug configured to limit a flow rate of the inflation fluid.

Clause 9. The cervical dilation device of clause 1, wherein the telescoping inner shaft has a distal portion bonded to the distal balloon end and a proximal portion freely slidable within the outer shaft portion, such that expansion of the inflatable dilation balloon controls an amount of telescoping movement.

Clause 10. The cervical dilation device of clause 1, further comprising a shutoff valve in fluid communication with the fluid lumen and configured to selectively block or permit delivery of the inflation fluid to the inflatable dilation balloon.

Clause 11. The cervical dilation device of clause 1, further comprising one or more of a distal balloon and an external reservoir.

Clause 12. The cervical dilation device of clause 1, further comprising one or more filling ports.

Clause 13. The cervical dilation device of clause 1, wherein the inflatable dilation balloon delivers a compound or composition topically.

Clause 14. A method of dilating a cervix of a patient, comprising:

inserting the cervical dilation device of clause 1 transvaginally such that the dilation balloon is positioned at least partially within a cervical canal;

delivering an inflation fluid from a proximal fluid reservoir region through the flow restrictor and into the dilation balloon, thereby gradually expanding the dilation balloon to dilate the cervical canal over time; and

maintaining the dilation balloon in an inflated state for a predetermined period, and thereafter deflating and removing the cervical dilation device from the patient.

Clause 15. The method of dilating the cervix of clause 14, wherein the inflation fluid is delivered at a controlled flow rate determined at least in part by the flow restrictor to reduce cramping or pain perceived by the patient.

Clause 16. The method of dilating the cervix of clause 14, further comprising positioning the dilation balloon such that, upon inflation, the balloon exerts compressive force on a uterus to reduce postpartum hemorrhage.

Clause 17. The method of dilating the cervix of clause 14, further comprising monitoring an inflation pressure of the dilation balloon using a pressure gauge or predetermined inflation volume.

Clause 18. A method of manufacturing a cervical dilation device, comprising:

providing an outer tubular member having a proximal end and a distal end;

providing an inner tubular member sized to telescope within at least a distal portion of the outer tubular member to form a telescoping inner shaft assembly;

bonding a distal portion of the inner tubular member to a distal section of an inflatable dilation balloon and bonding a proximal section of the dilation balloon to the outer tubular member such that the dilation balloon spans between the inner tubular member and the outer tubular member;

forming a fluid lumen extending from a proximal connector toward the dilation balloon; and

incorporating a flow restrictor into fluid communication between a proximal fluid reservoir region and the dilation balloon to control a rate of fluid flow into the dilation balloon.

Clause 19. The method of manufacturing a cervical dilation device of clause 18, wherein the dilation balloon is formed of a non‑compliant polymeric material and is pleated prior to assembly to facilitate delivery through the cervical canal.

Clause 20. The method of manufacturing a cervical dilation device of clause 18, further comprising selecting dimensional tolerances of the dilation balloon, telescoping inner shaft, and outer tubular member to maintain a predetermined annular clearance for passage of inflation fluid and telescoping movement.

Clause 21. The method of manufacturing a cervical dilation device of clause 18, further comprising bonding a proximal connector configured as a Luer connector to the proximal end of the outer tubular member.

Clause 22. A kit comprising:

a cervical dilation device according to clause 1; and

written instructions for use describing insertion of the cervical dilation device into a cervical canal, inflation of the dilation balloon to dilate the cervical canal and/or compress a uterus, and removal of the cervical dilation device.

Clause 23. The kit of clause 22, further comprising at least one syringe or inflation source configured to deliver a predetermined volume of inflation fluid to the dilation balloon.

Clause 24. The kit of clause 22, further comprising a pressure gauge or other indicator configured to assist a user in monitoring pressure or volume of the inflation fluid within the dilation balloon.

Clause 25. A method of manufacturing a cervical dilation device, comprising:

providing an outer tubular member having a proximal end and a distal end; providing an inner tubular member sized to telescope within at least a distal portion of the outer tubular member to form a telescoping inner shaft assembly; forming at least one fluid lumen extending along the outer tubular member from the proximal end toward a distal region of the outer tubular member; forming a plurality of fill ports in fluid communication with the at least one fluid lumen; and

bonding an inflatable dilation balloon to the outer tubular member at a proximal balloon bond and to a distal tubular member at a distal balloon bond such that the inflatable dilation balloon is in fluid communication with the at least one fluid lumen and spans between the proximal and distal balloon bonds.

Clause 26. The method of clause 25, wherein bonding the inflatable dilation balloon comprises thermally bonding ends of the dilation balloon to the outer tubular member and the distal tubular member and applying an adhesive to reinforce the balloon bonds.

Clause 27. The method of clause 25, further comprising pleating the dilation balloon prior to bonding or packaging to form a reduced profile configuration configured to facilitate insertion through a cervical canal.

Clause 28. The method of clause 25, wherein providing the inner tubular member comprises selecting dimensions of the inner tubular member and the outer tubular member to define an annular clearance therebetween that permits both axial telescoping movement and fluid flow toward the dilation balloon.

Clause 29. The method of clause 25, further comprising positioning at least one radiopaque marker band on the outer tubular member at a selected location beneath or adjacent the dilation balloon and securing the at least one radiopaque marker band with adhesive or over molding before bonding the dilation balloon.

Although these teachings have been described with respect to various examples, it should be appreciated that these teachings are also capable of a wide variety of further and other examples within the spirit and scope of these teachings. Thus, although an overview of the subject matter has been described with reference to specific examples, various modifications and changes may be made to these examples without departing from the broader scope of the present disclosure. It will also be obvious to those skilled in the art that the examples are provided by way of example only. Numerous variations, changes, and substitutions will occur to those skilled in the art without departing from the scope of the disclosure. It should also be understood that various alternatives may be employed in practicing the disclosed innovation. It is intended that any claims presented at any time in this application, or a subsequent application, define the scope of the invention and that methods and structures within the scope of any claims and their equivalents are covered thereby.

What is claimed:

1. A cervical dilation device comprising:
an elongated flexible tubular member having a proximal end and a distal end with a fluid channel;
an inflatable dilation balloon disposed adjacent the distal end of the elongated flexible tubular member, the inflatable dilation balloon having a proximal balloon end and a distal balloon end and being configured to expand to a diameter of up to about 30 mm;
a fluid lumen extending from the proximal end toward the inflatable dilation balloon;
a telescoping inner shaft disposed within an outer shaft portion of the elongated flexible tubular member, the telescoping inner shaft configured to translate relative to the outer shaft portion and resist kinking of the inflatable dilation balloon when the dilation balloon is subjected to tension; and
a flow restrictor fluidly coupled between a proximal fluid reservoir region and the inflatable dilation balloon and configured to control a rate of fluid flow into the inflatable dilation balloon.

2. The cervical dilation device of claim 1, wherein the telescoping inner shaft comprises an inner tube and an outer tube defining an annular clearance therebetween to permit passage of inflation fluid toward the inflatable dilation balloon.

3. The cervical dilation device of claim 1, wherein the inflatable dilation balloon is formed of a non‑compliant polymeric material selected from nylon, polyamide, polyethylene, polyurethane, or combinations thereof.

4. The cervical dilation device of claim 1, wherein the inflatable dilation balloon is configured to withstand an inflation pressure of at least about 4 atmospheres.

5. The cervical dilation device of claim 1, further comprising a proximal connector configured to couple to a syringe or other fluid source to deliver inflation fluid to the inflatable dilation balloon.

6. The cervical dilation device of claim 1, wherein the elongated flexible tubular member has a curved distal portion configured to facilitate insertion through a cervical canal.

7. The cervical dilation device of claim 1, wherein the inflatable dilation balloon is configured, in a first mode of operation, to dilate a cervical canal over a period of time and, in a second mode of operation, to compress a uterus to treat postpartum hemorrhage.

8. The cervical dilation device of claim 1, wherein the flow restrictor comprises a narrowed lumen region, orifice, valve, or porous plug configured to limit a flow rate of the inflation fluid.

9. The cervical dilation device of claim 1, wherein the telescoping inner shaft has a distal portion bonded to the distal balloon end and a proximal portion freely slidable within the outer shaft portion, such that expansion of the inflatable dilation balloon controls an amount of telescoping movement.

10. The cervical dilation device of claim 1, further comprising a shutoff valve in fluid communication with the fluid lumen and configured to selectively block or permit delivery of the inflation fluid to the inflatable dilation balloon.

11. The cervical dilation device of claim 1, further comprising one or more of a distal balloon and an external reservoir.

12. The cervical dilation device of claim 1, further comprising one or more filling ports.

13. The cervical dilation device of claim 1, wherein the inflatable dilation balloon delivers a compound or composition topically.

14. A method of dilating a cervix of a patient, comprising:
inserting the cervical dilation device of claim 1 transvaginally such that the dilation balloon is positioned at least partially within a cervical canal;
delivering an inflation fluid from a proximal fluid reservoir region through the flow restrictor and into the dilation balloon, thereby gradually expanding the dilation balloon to dilate the cervical canal over time; and
maintaining the dilation balloon in an inflated state for a predetermined period, and thereafter deflating and removing the cervical dilation device from the patient.

15. The method of dilating the cervix of claim 14, wherein the inflation fluid is delivered at a controlled flow rate determined at least in part by the flow restrictor to reduce cramping or pain perceived by the patient.

16. The method of dilating the cervix of claim 14, further comprising positioning the dilation balloon such that, upon inflation, the balloon exerts compressive force on a uterus to reduce postpartum hemorrhage.

17. The method of dilating the cervix of claim 14, further comprising monitoring an inflation pressure of the dilation balloon using a pressure gauge or predetermined inflation volume.

18. A method of manufacturing a cervical dilation device, comprising:
providing an outer tubular member having a proximal end and a distal end;
providing an inner tubular member sized to telescope within at least a distal portion of the outer tubular member to form a telescoping inner shaft assembly;
bonding a distal portion of the inner tubular member to a distal section of an inflatable dilation balloon and bonding a proximal section of the dilation balloon to the outer tubular member such that the dilation balloon spans between the inner tubular member and the outer tubular member;
forming a fluid lumen extending from a proximal connector toward the dilation balloon; and
incorporating a flow restrictor into fluid communication between a proximal fluid reservoir region and the dilation balloon to control a rate of fluid flow into the dilation balloon.

19. The method of manufacturing a cervical dilation device of claim 18, wherein the dilation balloon is formed of a non‑compliant polymeric material and is pleated prior to assembly to facilitate delivery through the cervical canal.

20. The method of manufacturing a cervical dilation device of claim 18, further comprising selecting dimensional tolerances of the dilation balloon, telescoping inner shaft, and outer tubular member to maintain a predetermined annular clearance for passage of inflation fluid and telescoping movement.

21. The method of manufacturing a cervical dilation device of claim 18, further comprising bonding a proximal connector configured as a Luer connector to the proximal end of the outer tubular member.

22. A kit comprising:
a cervical dilation device according to claim 1; and
written instructions for use describing insertion of the cervical dilation device into a cervical canal, inflation of the dilation balloon to dilate the cervical canal and/or compress a uterus, and removal of the cervical dilation device.

23. The kit of claim 22, further comprising at least one syringe or inflation source configured to deliver a predetermined volume of inflation fluid to the dilation balloon.

24. The kit of claim 22, further comprising a pressure gauge or other indicator configured to assist a user in monitoring pressure or volume of the inflation fluid within the dilation balloon.

25. A method of manufacturing a cervical dilation device, comprising:
providing an outer tubular member having a proximal end and a distal end;
providing an inner tubular member sized to telescope within at least a distal portion of the outer tubular member to form a telescoping inner shaft assembly;
forming at least one fluid lumen extending along the outer tubular member from the proximal end toward a distal region of the outer tubular member;
forming a plurality of fill ports in fluid communication with the at least one fluid lumen; and
bonding an inflatable dilation balloon to the outer tubular member at a proximal balloon bond and to a distal tubular member at a distal balloon bond such that the inflatable dilation balloon is in fluid communication with the at least one fluid lumen and spans between the proximal and distal balloon bonds.

26. The method of claim 25, wherein bonding the inflatable dilation balloon comprises thermally bonding ends of the dilation balloon to the outer tubular member and the distal tubular member and applying an adhesive to reinforce the balloon bonds.

27. The method of claim 25, further comprising pleating the dilation balloon prior to bonding or packaging to form a reduced profile configuration configured to facilitate insertion through a cervical canal.

28. The method of claim 25, wherein providing the inner tubular member comprises selecting dimensions of the inner tubular member and the outer tubular member to define an annular clearance therebetween that permits both axial telescoping movement and fluid flow toward the dilation balloon.

29. The method of claim 25, further comprising positioning at least one radiopaque marker band on the outer tubular member at a selected location beneath or adjacent the dilation balloon and securing the at least one radiopaque marker band with adhesive or over molding before bonding the dilation balloon.
